(19) United States
(12) Patent Application Publication
(10) Pub. No.: US 2018/0042591 A1
(43) Pub. Date: Feb. 15, 2018

(54) SURGICAL INSTRUMENTS INCLUDING ADJUSTABLE HANDLE ASSEMBLIES

(71) Applicant: Covidien LP, Mansfield, MA (US)

(72) Inventors: Mark Russo, Plantsville, CT (US); Jin Yong, Watertown, CT (US); Jessica Dinan, New Haven, CT (US); Michael Kolb, Southington, CT (US); Kevin Sniffin, Roxbury, CT (US); Brian Laird, Granby, CT (US)

(21) Appl. No.: 15/677,277

(22) Filed: Aug. 15, 2017

Related U.S. Application Data
(60) Provisional application No. 62/375,025, filed on Aug. 15, 2016.

(51) Int. Cl. A61B 17/00

(52) U.S. Cl. CPC A61B 17/00; A61B 2017/00424; A61B 2017/00438; A61B 2017/0046; A61B 2017/00477; A61B 17/064

(57) ABSTRACT

A handheld surgical instrument is provided, including a housing, a handle and a trigger mounted on the housing, and an elongated tubular portion extending distally from the housing. The surgical instrument includes a hand grip having a top surface, a bottom surface, and at least one opening disposed therebetween, wherein the handle of the handheld surgical instrument is insertable into the at least one opening of the hand grip for engagement therewith. When the hand grip is engaged to the handle of the handheld surgical instrument, the hand grip affects a distance defined between the handle and the trigger.

### CROSS-REFERENCE TO RELATED APPLICATIONS

This application claims the benefit of and priority to U.S. Provisional Patent Application No. 62/375,025 filed Aug. 15, 2016, the entire disclosure of which is incorporated by reference herein.

### BACKGROUND

### 1. Technical Field

The present disclosure generally relates to surgical instruments and, more particularly, to improved handle assemblies for surgical instruments.

### 2. Background of Related Art

Handheld surgical instruments, e.g., electrosurgical forceps, shears, tack appliers, staplers, are well known in the medical arts and typically include a housing, a handle assembly, a shaft extending from the handle assembly, and in some instruments, an end effector assembly attached to a distal end of the shaft. The handle assembly may include one or more fixedly attached hand grips that provide a gripping surface for an operator, e.g., a clinician, such that the operator may securely grasp the surgical instrument.

A feature of these surgical instruments is that the handle assemblies are “one size fits all” and not custom designable for operators with hands that are outside the range of average sizes, or for operators having a left or right hand preference.

Accordingly, a need exists for ergonomically designed handles that can accommodate operators of all hand sizes to enhance comfort and grip during surgical procedures.

### SUMMARY

The present disclosure relates to adjustable handle assemblies for use with surgical instruments.

According to an aspect of the present disclosure, a handheld surgical instrument is provided, including a housing, a handle and a trigger mounted on the housing, and an elongated tubular portion extending distally from the housing. The surgical instrument includes a hand grip having a top surface, a bottom surface, and at least one opening disposed therebetween, wherein the handle of the handheld surgical instrument is insertable into the at least one opening of the hand grip for engagement therewith, and wherein when the hand grip is engaged to the handle of the handheld surgical instrument, the hand grip affects a distance defined between the handle and the trigger.

The hand grip may be selectively engageable with the trigger of the handheld surgical instrument. When the hand grip is engaged with the trigger of the handheld surgical instrument, the hand grip affects the distance defined between the handle and the trigger.

The hand grip may further include finger grips for placement of fingers therein.

The hand grip may have at least one protuberance or at least one indentation to enhance grip.

The at least one opening may be a cavity extending through the top surface of the hand grip to the bottom surface of the hand grip. The at least one opening may be snap fitted with a back portion of the handle. The at least one opening may be a slot extending from the top surface of the hand grip to the bottom surface of the hand grip; and wherein the slot slidably receives a protrusion disposed along the length of the handle of the handheld instrument.

According to another aspect of the present disclosure, a handheld surgical instrument is provided, including a housing, a handle movably mounted on the housing, an elongated tubular portion extending distally from the housing, and a trigger movably mounted on the housing.

The handle may be pivotably connected to the housing such that the handle can pivot about the housing from a first position to a second position.

The handle may include an inner handle fixedly mounted to the housing, an outer handle rotatably mounted on the inner handle, the outer handle having a non-uniform shape, and wherein the outer handle rotates about the inner handle from a first gripping position to a second gripping position.

The handheld surgical instrument may further include a hand grip including a hand grip body having a top surface, a bottom surface, and at least one opening disposed therebetween, wherein the handle of the handheld surgical instrument is insertable into the at least one opening of the hand grip for engagement therewith, and wherein when the hand grip is engaged to the handle of the handheld surgical instrument, the hand grip affects a distance defined between the handle and a trigger.

The hand grip may be selectively engageable with the trigger of the handheld surgical instrument. When the hand grip is engaged to the trigger of the handheld surgical instrument, the hand grip affects the distance defined between the handle and the trigger.

According to yet another aspect of the present disclosure, a handheld surgical instrument is provided, including a handpiece, a shaft assembly extending distally from the handpiece, and a hand grip slidably disposed over the handpiece, wherein the hand grip is movable from a proximal end portion of the handpiece to a distal end portion of the handpiece.

According to still yet another aspect of the present disclosure, a handheld surgical instrument kit is provided, including a handheld surgical instrument, including a housing, a handle and a trigger mounted on the housing and an elongated tubular portion extending distally from the housing. At least two hand grips may also be included, each having a top surface, a bottom surface, and at least one opening disposed therebetween, wherein each hand grip is engageable with a handle of the handheld surgical instrument, and wherein when a selected one of the at least two hand grips is engaged to the handle of the handheld surgical instrument, the selected hand grip affects a distance defined between the handle and the trigger.

At least one of the at least two hand grips may be engageable with the trigger of the handheld surgical instrument.

### BRIEF DESCRIPTION OF THE DRAWINGS

FIG. 1 is a perspective view of a surgical instrument;

FIG. 2 is a side view, with a housing half removed, of the housing portion of the surgical instrument in an initial position;

FIG. 3 is a perspective view, partially shown in cross-section, of a distal end portion of the surgical instrument corresponding to FIG. 1;

FIG. 4 is a side view, with a housing half removed, of the housing portion of the surgical instrument in an actuated position;

FIG. 5 is a schematic illustration of an embodiment of a hand grip shown in relation to a handle of the surgical instrument of FIG. 1;

FIG. 6 is a perspective schematic illustration of another embodiment of a hand grip shown in relation to the handle of the surgical instrument of FIG. 1;

FIG. 7 is a schematic illustration of yet another embodiment of a hand grip shown in relation to a modified handle of the surgical instrument of FIG. 1;

FIG. 8 is a schematic illustration of an embodiment of a trigger grip shown in relation to a trigger of the surgical instrument of FIG. 1;

FIG. 9 is a schematic illustration of another embodiment of a surgical instrument in accordance with the present disclosure;

FIG. 10 is a schematic illustration of yet another embodiment of a surgical instrument in accordance with the present disclosure;

FIG. 11 is a schematic illustration of still another embodiment of a surgical instrument in accordance with the present disclosure;

FIG. 12 is a schematic illustration of still yet another embodiment of a surgical instrument in accordance with the present disclosure;

FIG. 13 is a schematic illustration of another embodiment of a surgical instrument in accordance with the present disclosure; and

FIG. 14 is a schematic illustration of a surgical instrument kit in accordance with the present disclosure.

### DETAILED DESCRIPTION

The present disclosure is directed to improved handle assemblies for handheld surgical instruments. Specifically, attachable, adjustable, and interchangeable ergonomic handle assemblies that may be positioned on the handle and/or trigger portions of handheld surgical instruments are provided herein. The handle assemblies are configured to accommodate operators of all hand sizes to enhance comfort, grip, operating efficiency, or the like during surgical procedures.

As shown in the drawings and described throughout the following description, as is traditional when referring to relative positioning on a surgical instrument, the term “proximal” refers to the end of the instrument which is closer to the operator and the term “distal” refers to the end of the instrument which is further away from the operator. Directional reference terms, such as “top,” “bottom,” “back,” “front,” “side,” and the like, are intended to ease description of the embodiments and are not intended to have any limiting effect on the ultimate orientations of the surgical devices, or any parts thereof.

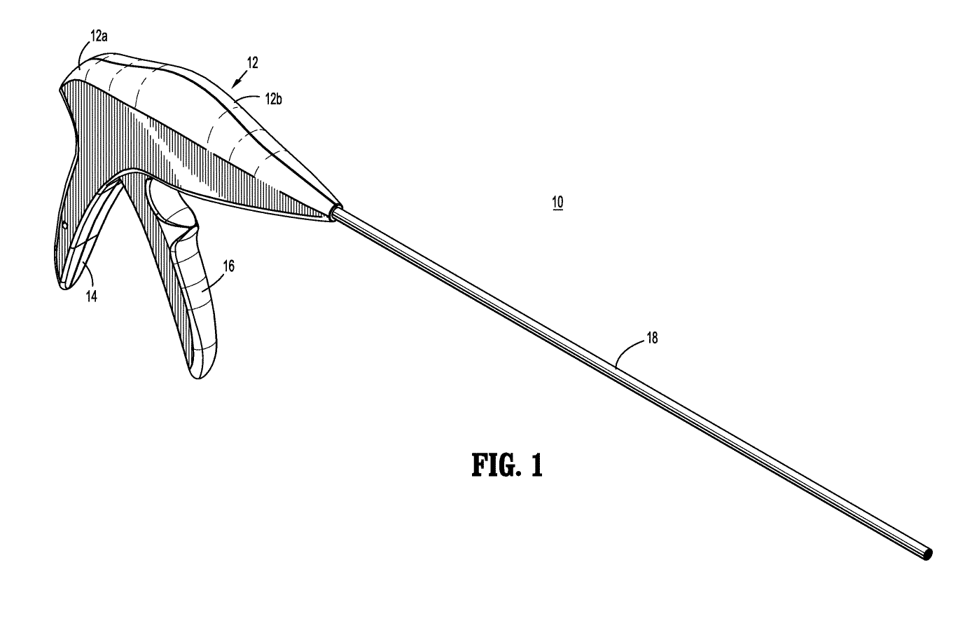

As shown in FIG. 1, a surgical instrument, in accordance with an embodiment of the present disclosure is generally designated by reference numeral 10. Although surgical instrument 10 is shown and described as a tack applier herein (e.g., for the sake of simplicity), it should be appreciated that the handle assemblies of the present disclosure may be used with any type of handheld surgical instrument, e.g., motorized and non-motorized electrosurgical forceps, shears, clip appliers, staplers, aspirators, ablation probes, or the like. In addition, aspects and features of surgical instrument 10 not germane to the understanding of the present disclosure are omitted to avoid obscuring the aspects and features of the present disclosure in unnecessary detail. For a more detailed description of the construction and operation of an example of a tack applier, reference may be made to U.S. Pat. No. 5,830,221, the entire content of which is incorporated herein by reference.

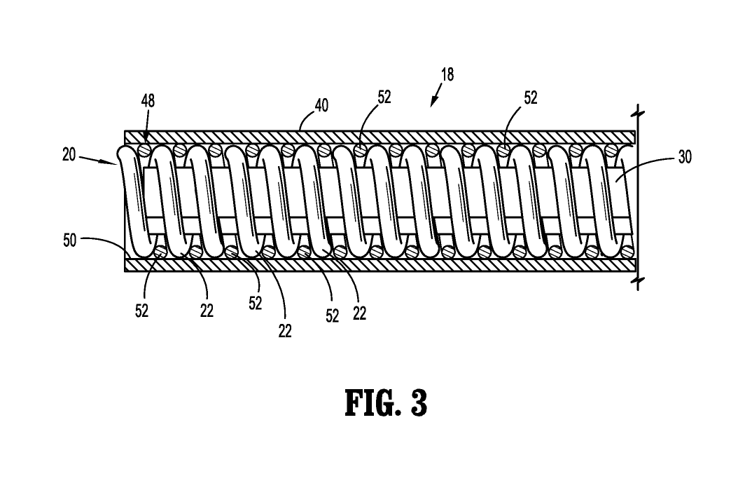

Surgical instrument 10 is provided to apply fasteners to tissue or to secure mesh to tissue during surgical procedures such as e.g., hernia repair. Surgical instrument 10 generally includes a housing 12 and a handle portion 14 extending from housing 12. A trigger 16 is movably mounted to housing 12. Trigger 16 is pivotally connected to housing 12 with a free end of trigger 16 spaced from a free end of handle portion 14. Surgical instrument 10 also includes an elongated tubular portion 18 extending distally from housing 12. The elongated tubular portion 18 is provided to retain, e.g., a plurality of coil fasteners 20 (FIG. 3) for application to body tissue. In operation, as trigger 16 is actuated, a fastener 20 (FIG. 3) is fired from elongated tubular portion 18 to secure a surgical mesh to tissue.

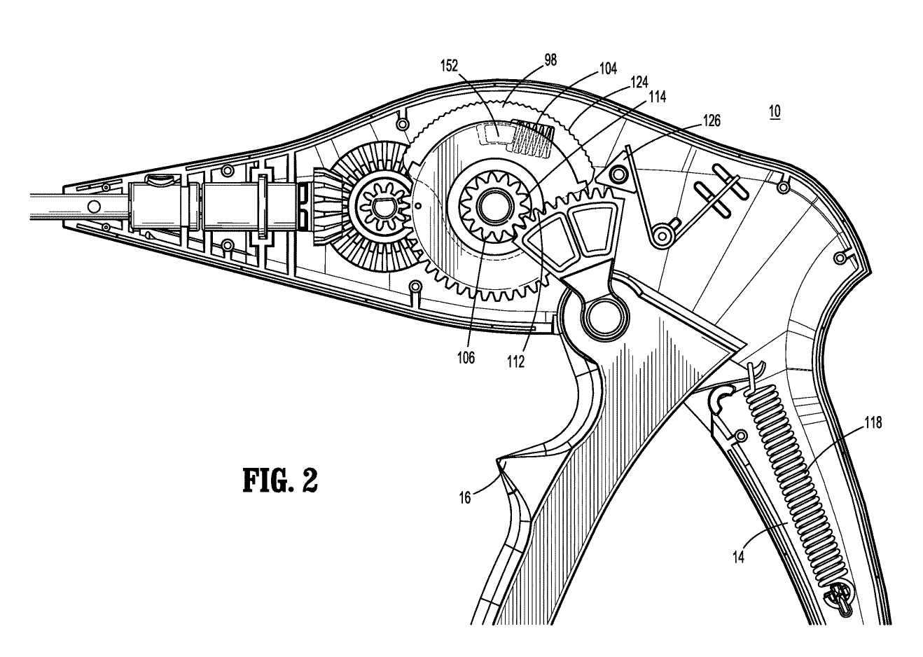

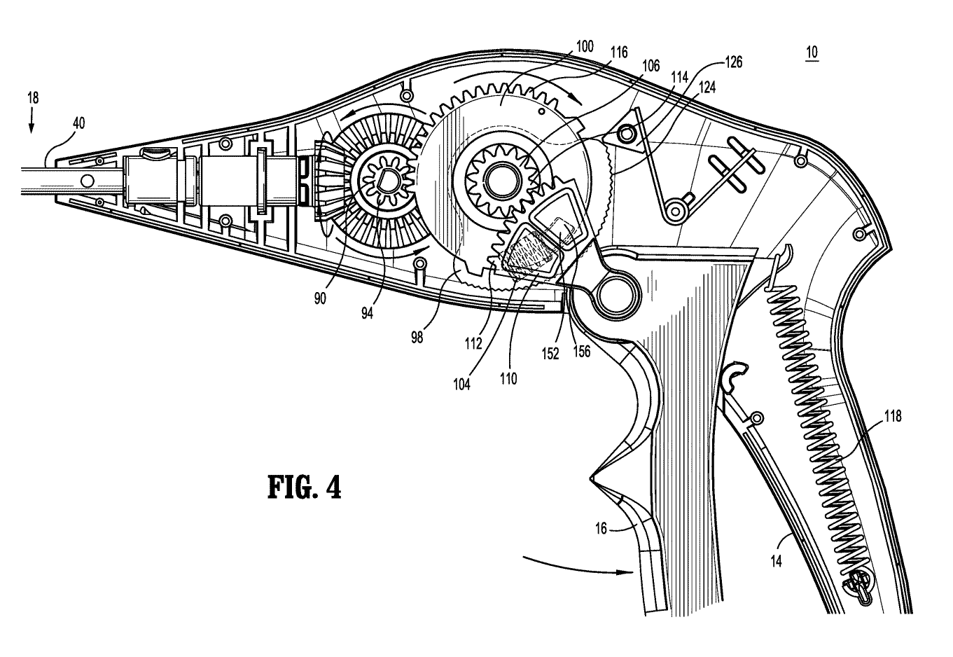

Referring now to FIG. 2, in an initial or starting position, trigger 16 is biased away from handle 14 due to the force of return spring 118. As shown, teeth 112 of trigger 16 are engaged with teeth 114 of trigger gears 106, ratchet-plate gear 98 is in a counterclockwise most position, and pawl 126 is disengaged from teeth 124 of ratchet-plate gear 98. Referring now to FIG. 3, within distal end 46 of tubular sleeve 40, a plurality of coil fasteners 20 are slidably mounted about drive rod 30, all of which are disposed within elongated tubular shaft 18. Each coil body portion 22 of each coil fastener 20 engages surface 52 of coil spring 48, which is firmly secured to inner surface 50 of tubular sleeve 40. Referring now to FIG. 4, to actuate surgical instrument 10, trigger 16 is drawn toward handle 14 against the bias of return spring 118. As trigger 16 is moved, teeth 112 on gear portions 110 of trigger 16 engage and rotate teeth 114 of trigger gears 106 clockwise. Rotation of trigger gears 106 rotates ratchet-plate gear 98 such that first edge 156 of engagement tab 152 engages ratchet-plate gear 98. Idler gear 100 thus rotates with ratchet-plate gear 98 allowing a short expansion of compression spring 104. As idler gear 100 is rotated in a clockwise direction, as viewed in FIG. 4, teeth 116 of idler gear 100 engage and rotate drive gear teeth 94 of drive gear 90 counterclockwise.

With continued reference to FIG. 4, upon a complete depression of trigger 16, drive rod 30 (FIG. 3) is rotated precisely a predetermined amount such that only one coil fastener 20 is driven out of the distal end of elongated tubular portion 18. During compression of trigger 16, pawl 126 engages and rides over teeth 124 of ratchet-plate gear 98. Should handle 16 be stopped during depression at any intermediate position, pawl 126 is engaged with teeth 124 to ensure that ratchet-plate gear 98 and idler gear 100 are not rotated in an opposite direction thereby preventing only partial insertion or withdrawal of coil fastener 20, i.e. preventing a partial drive cycle. As trigger 16 is completely depressed, pawl 126 passes over teeth 124 and is disengaged therefrom.

As can be appreciated, it is often necessary to actuate surgical instrument 10 several times during a single procedure. Additionally, it may be necessary to use a large amount of force, e.g., on handle portion 14 and trigger 16 for extended periods during certain procedures. The hands of different operators come in a variety of shapes and sizes and each operator may have a left or right hand preference. As will be described below, handle assemblies and surgical instruments of the present disclosure provide enhanced comfort and grip for any hand size or dexterity.

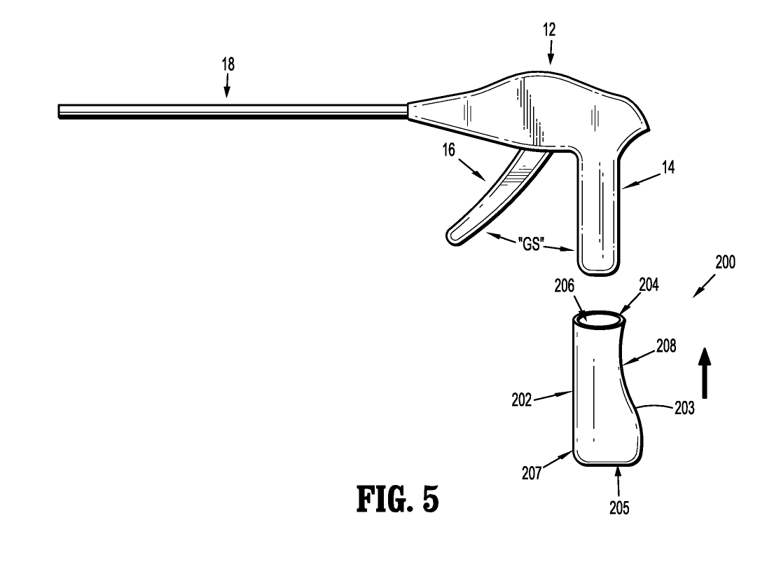

Referring now to FIG. 5, a handle grip 200 of the present disclosure is shown. As will be described in more detail below, handle grip 200 is positioned on and/or over handle portion 14 of surgical instrument 10 to provide operators, e.g., clinicians of any hand size or preference with enhanced comfort and grip during surgical procedures. Handle grip 200 generally includes a distal wall 202, a proximal wall 203, an upper portion 204, a lower portion 205, a cavity 206 and left sidewall 206, right sidewall 207, and a cavity 208 defined by walls 202, 203, 206, and 207. Handle grip 200 may be made or molded of any suitable material including plastic, metal or metal alloy, rubber, or the like. Handle grip 200 may be rigid, semi-rigid, or flexible. As can be appreciated, handle grip 200 may be made or sized to accommodate any hand size (e.g., small, medium, and large), or may be formed or molded to a specific operator's hand and/or hand preference (e.g., left or right).

With continued reference to FIG. 5, distal wall 202 of handle grip 200, as shown, has a substantially straight, flat, or planar profile to accommodate the fingers and/or the metacarpo-phalangeal joints of the hand. Proximal wall 203 of handle grip 200, as shown, has a substantially contoured profile for the thumb and palm portion of the hand. As can be appreciated, distal wall 202 and proximal wall 203 of handle grip 200, as well as left sidewall 206 and right sidewall 207 of handle grip 200, may have any suitable shape, profile, material or other property to best provide comfort and grip to an operator. For example, handle grip 200 may have a series of finger pads, indentations, or divots for resting fingers therein. As another example, handle grip 200 may have a protrusion or a series of protrusions (not shown), such as an aggressive knurled pattern or checkering disposed thereon to ensure maximum grip and comfort.

Handle grip 200 operably couples to handle portion 14 via one or more suitable coupling methods. As shown in FIG. 5, handle portion 14 of surgical instrument 10 may be inserted into cavity 206 of handle grip 200 until upper portion 204 of handle grip 200 is in abutment with housing 12 and/or the lower portion of handle portion 14 is proximal with the lower portion 205 of handle grip 200. Cavity 206 of handle grip 200 may be coupled to handle portion 14 by, e.g., friction fit, snap-fit, press-fit, low pressure adhesive, or the like. Thus, by selecting an appropriate handle grip 200 based on the operator's hand size, an operator may achieve a desired grip span “GS” (e.g., an appropriate distance between handle portion 14 and trigger 16) for e.g., a desired level of comfort and grip.

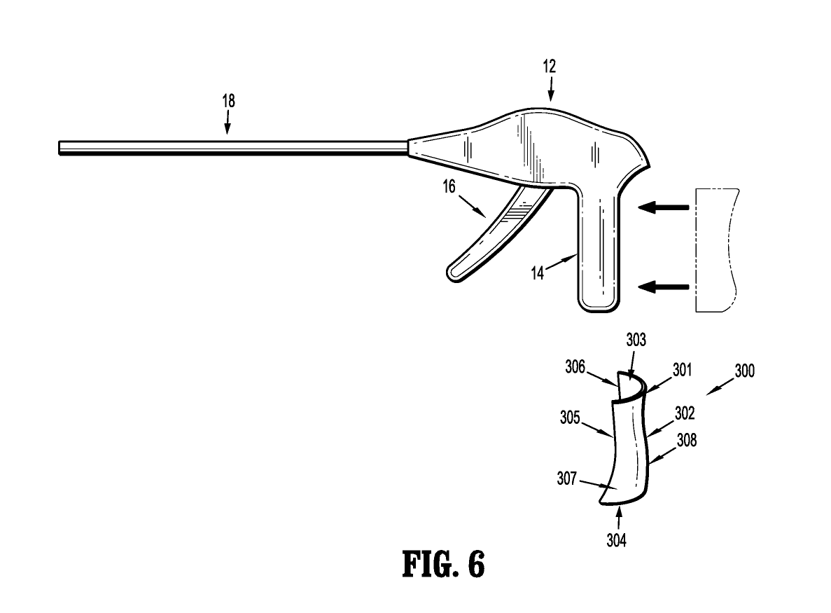

Another embodiment of a handle grip for use with surgical instrument 10 is shown in FIG. 6 and generally designated as 300. Snap handle grip or back-strap 300 generally includes a body 301, a proximal surface 302, an upper portion 303, a lower portion 304, left sidewall 305, right sidewall 306, an opening 307, and a cavity 308 defined by left sidewall 305 and right sidewall 306. Opening 305 of snap handle grip 300 may extend from upper portion 303 to lower portion 304. Snap handle grip 300 may be operably coupled to handle portion 14 by way of snap fit, such that snap handle grip 300 may be easily installed onto, and/or removed from, handle portion 14 of surgical instrument 10. For example, an operator may position opening 307 of snap handle grip 300 such that the length of snap handle grip 300 aligns with handle portion 14 of surgical instrument 10. An operator then applies a lateral force to snap handle grip 300 to push it onto handle portion 14, such that snap handle 300 is snapped into position on handle portion 14. Alternatively, snap handle grip 300 may be coupled to handle portion 14 of surgical instrument 10 using a press fit, friction fit, low pressure adhesive, or the like. Snap handle grip 300 may share some of or all of the same materials and/or properties described in the embodiment of handle grip 200 above.

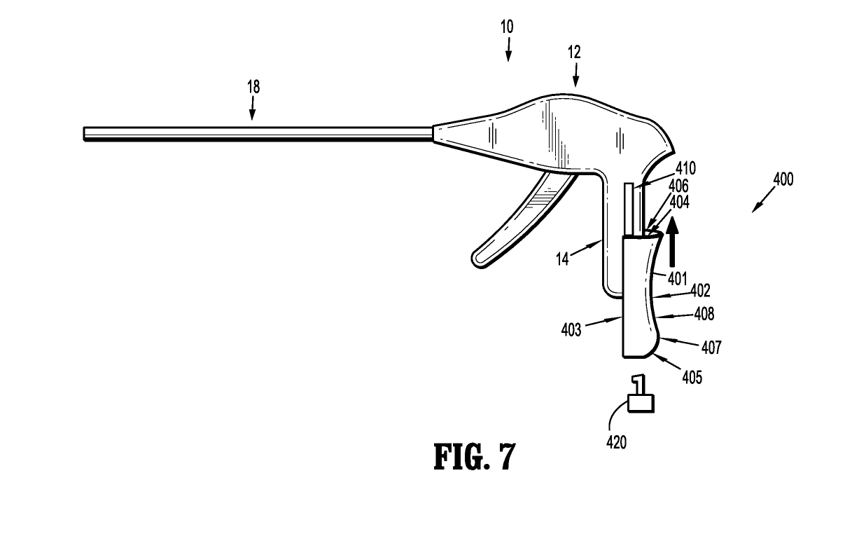

As an additional example, with reference to FIG. 7, a sliding handle grip for use with surgical instrument 10 and to, e.g., accommodate different hand sizes, is shown and generally designated as 400. Sliding handle grip 400 generally includes a body 401, a proximal wall 402, a distal wall 403, an upper portion 404, a lower portion 405, left sidewall 406, right sidewall 407, and an indentation 408 disposed on distal wall 403 and defined by left sidewall 406 and right sidewall 407. Specifically, sliding handle grip 400 is designed for slidable engagement with handle portion 14 of surgical instrument 10. In the alternative embodiment shown in FIG. 7, handle portion 14 of surgical instrument 10 may have a raised protrusion or slotted portion 410 running along the entire length of handle portion 14, which engages with a slot or indentation 408 of sliding handle grip 400 such that sliding handle grip 400 may be slid onto and coupled to handle portion 14 of surgical instrument 10. Indentation 406 of distal wall 403 may be disposed at least a portion or along the entire length of sliding handle grip 400 for engagement with slotted portion 410 of handle portion 14.

In order to position sliding handle grip 400 onto handle portion 14 of surgical instrument 10, an operator positions sliding handle grip 400 underneath handle portion 14, such that indentation 408 of upper portion 404 of handle grip 400 aligns and engages with slotted portion 410 of surgical instrument 10. An operator then applies an upward force onto sliding handle grip 400 until upper portion 404 thereof abuts with housing 12 of surgical instrument 10 and lower portion 405 thereof is flush with the bottom portion of handle portion 14. After sliding handle grip 400 is in place on handle portion 14, a locking pin 420 may then be inserted into a pin hole (not shown) on the lower portion 405 of sliding handle grip 400 to keep sliding handle grip 400 in place during use. Additionally or alternatively, sliding handle grip 400 may have a friction fit with raised slotted portion 410 of handle portion 14, such that sliding handle grip 400 remains affixed to handle portion 14 during use.

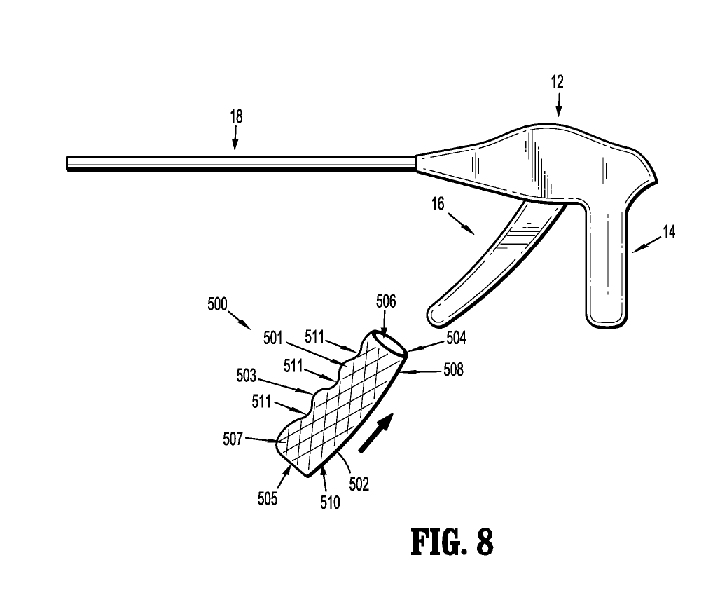

As an additional example, with reference to FIG. 8, a trigger grip for use with surgical instrument 10 to, e.g., accommodate different sized hands, is shown and generally designated as 500. Trigger grip 500 is designed for coupling to trigger 16 of surgical instrument 10. Trigger grip 500 may be used separately or in combination with any of the embodiments described herein. Trigger grip 500 generally includes a body 501, a proximal wall 502, a distal wall 503, an upper portion 504, a lower portion 505, left sidewall 506, right sidewall 507, and a cavity 508 defined by walls 502, 503, 506, and 507. Trigger grip 500 may be made or molded from a flexible and conformable material, e.g., rubber, plastic, or the like. As such, trigger grip 500 can be designed as a flexible insert. For example, trigger 14 of surgical instrument 10 may be inserted into cavity 508 of trigger grip 500. Additionally or alternatively, proximal wall 502 of trigger grip 500 may have a split seam 510, which allows left sidewall 506 and right side wall 507 to be spread apart or separated such that cavity 508 of trigger grip 500 can encapsulate and form over trigger 14 of surgical instrument 10. As can be appreciated, trigger grip 500 may be made to accommodate any hand size (e.g., small, medium, and large) or preference (e.g., left or right) or may be customized for a specific operator.

As shown in FIG. 8, distal wall 503 of trigger grip 500 may have finger grips 511 for placement of fingers therein. In accordance with the present disclosure, finger grips 511 may provide enhanced grip and comfort. For example, certain procedures may expose trigger 16 to bodily fluids, in which case finger grips 511 of trigger grip 500 may provide a clinician with enhanced comfort/grip. Additionally, body 501 of trigger grip 500 may have indentations, protrusions, or the like (e.g., embossing, knurled pattern, checkering, etc.) to further enhance grip.

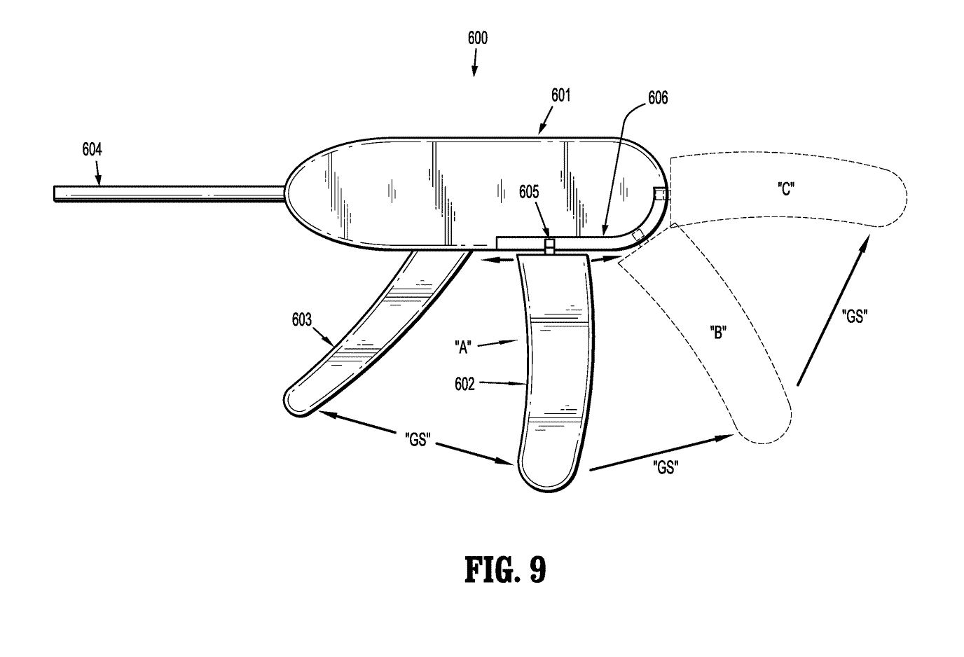

With reference to FIG. 9, another embodiment of a surgical instrument for use with surgical instrument 10 to, e.g., accommodate different sized hands, is shown and generally designated as 600. Surgical instrument 600 generally includes a housing 601, an adjustable handle portion 602, a trigger 603, and an elongated tubular shaft 604. Handle portion 602 may include a pin 605 located on an upper portion thereof and which may be slidably connected to and disposed within a slot 606 formed in a lower end of housing 601. Pin 605 in combination with slot 606 permits the slidable movement of adjustable handle portion 602 about housing 601 into e.g., positions “A,” “B,” and “C,” as shown in FIG. 9. For example, in position “A,” an operator may use surgical instrument 600 in a pistol grip configuration, while in position “C,” an operator may use surgical instrument 600, e.g., as a pencil-style device. Position “B” offers an intermediate position between positions “A” and “C.” Operators of different hand sizes and dexterity may use any of the positions during surgical procedures, e.g., for increased stability and/or advantage.

In use, a grip span “GS” of surgical instrument 600 is changed when adjustable handle portion 602 is moved, for example, from position “A” to “B,” “B” to “A,” “A” to “C,” “C” to “A,” “B” to “C,” or “C” to “B.” Adjustable handle portion 602 may be locked into and secured into any of the aforementioned positions (e.g., with the use of a ratcheting system or the like, not shown). It should be appreciated that any of the embodiments described herein may be used with surgical instrument 600. For example, any of the interchangeable handle or trigger grips described herein may be positioned onto handle portion 602 or trigger 603 of surgical instrument 600.

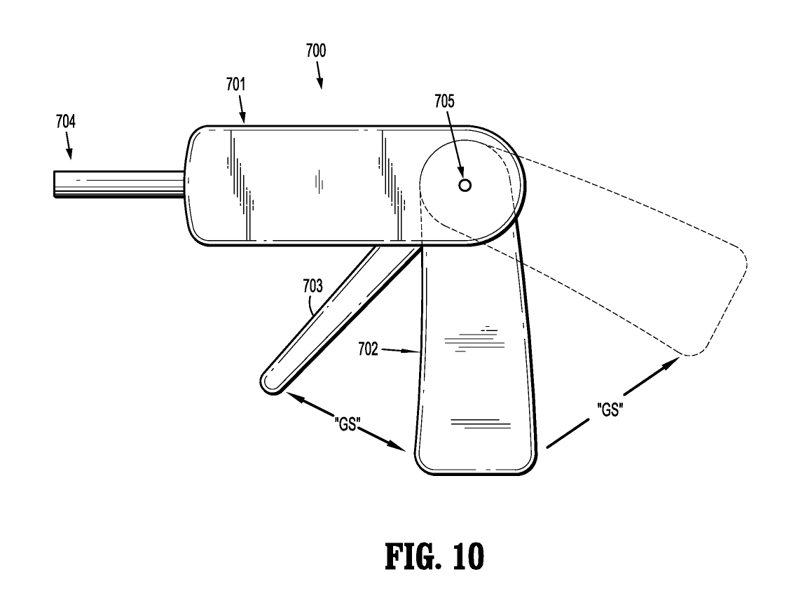

With reference to FIG. 10, another embodiment of a surgical instrument is shown and generally designated as 700. Surgical instrument 700 generally includes a housing 701, a pivotable handle portion 702, a trigger 703, and an elongated tubular shaft 704. Pivotable handle portion 702 is pivotably connected to housing 701 through a pivotable connection 705. Similar to the embodiment of surgical instrument 600 described above, pivotable handle portion 702 of surgical instrument 700 is movable and securable into several different positions to obtain a desired grip span “GS,” as shown in FIG. 10. Pivotable handle portion 702 of surgical instrument 700 also functions to change the angle of the axis of housing 701 and elongated tubular shaft 704 relative to the axis of pivotable handle portion 702. Trigger 703 may also pivot with, or separately from, pivotable handle portion 702.

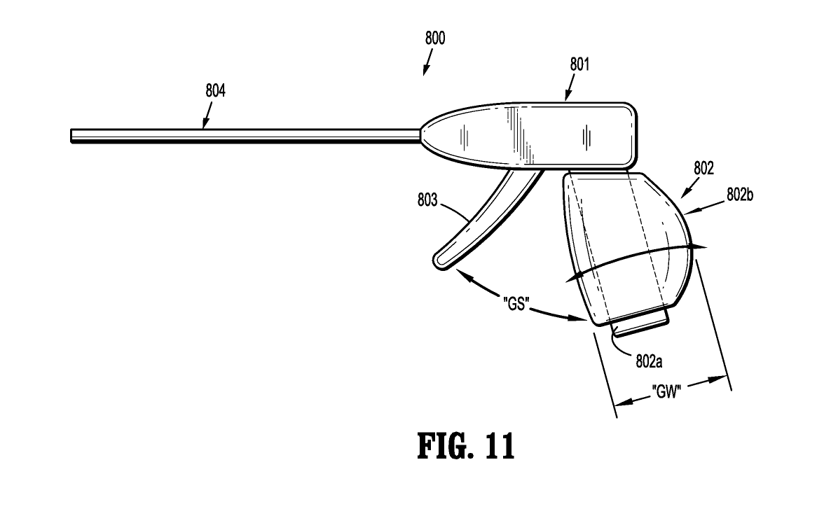

With reference to FIG. 11, another embodiment of a surgical instrument is shown and generally designated as 800. Surgical instrument 800 generally includes a housing 801, a handle assembly 802, a trigger 803, and an elongated tubular shaft 804. Handle assembly 802 includes an inner core or stem 802a and an outer sleeve 802b, which is rotatably connected to and may be coterminous with inner stem 802a. Inner handle 802a of handle assembly 802 may be a stationary spindle, or the like, with a ratcheting mechanism (e.g., a snap, or intent/detent mechanism, not shown) for allowing outer sleeve 802b to rotate about inner stem 802a, and lock or be secured into a desired position. Outer sleeve 802b may have a non-uniform, non-circular, or irregularly shaped profile such that when outer sleeve 802b is rotated about inner stem 802 of handle assembly 802, the grip width “GW” is adjusted to suit an operator's preference. As can be appreciated, a desired grip width “GW” affects the grip span “GS,” or the spacing between handle assembly 802 and trigger 803. Outer sleeve 802b may be formed from, for example, a soft, pliable, comformable, rigid or semi-rigid material. Outer sleeve 802b may have any number of indentations, protrusions, finger grips, or the like, for placing fingers therein. In addition, outer sleeve 802b may have at least one protrusion, indentation, or the like to provide enhanced grip and/or comfort.

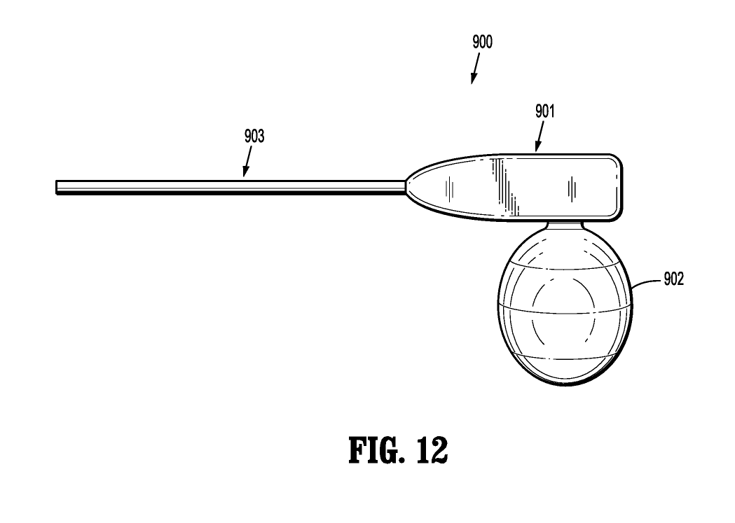

With reference to FIG. 12, another embodiment of a surgical instrument is shown and generally designated as 900. Surgical instrument 900 includes a housing 901, a bulb handle assembly 902, and an elongated tubular shaft 903. Bulb handle assembly 902 may be a flexible balloon, similar to the bulbs used in blood pressure cuffs. Bulb handle assembly 902 may act as both a handle and a trigger. For example, bulb handle assembly 902 may comprise a mechanism that converts air pressure to linear/rotational displacement to, e.g., eject a tack from shaft 903. The size of bulb handle assembly 902 may be varied by inflating and deflating bulb handle assembly 902 to accommodate different hand sizes.

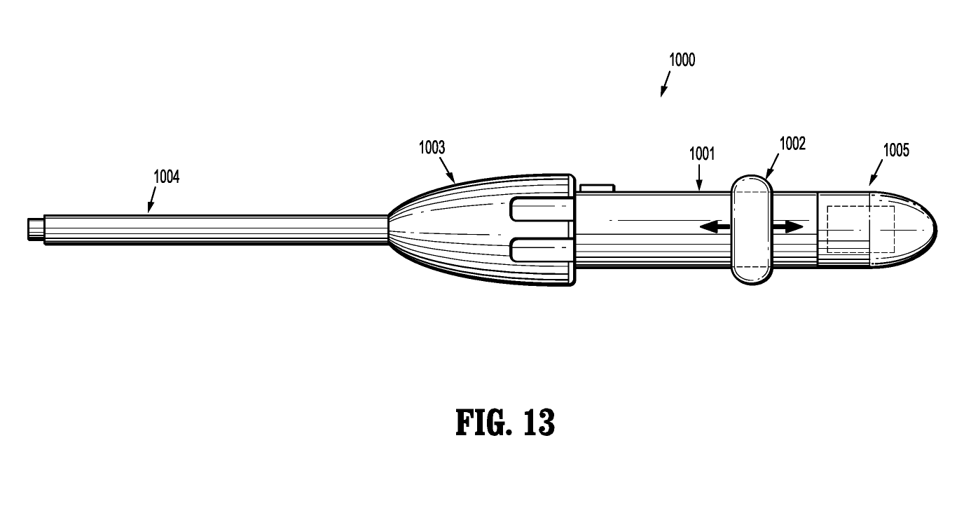

With reference to FIG. 13, another embodiment of a surgical instrument is shown and generally designated as 1000. Surgical instrument 1000 may be a handpiece or pencil style device. Surgical instrument 1000 generally includes a handpiece 1001, a hand grip 1002, an articulating knob 1003, and an elongated tubular shaft 1004. Articulation knob 1003 may be disposed at a distal end portion of the handpiece 1001 and operably coupled to elongated tubular shaft 1004 for selectively rotating elongated tubular shaft 1004 relative to handpiece 1001. Additionally or alternatively, surgical instrument 1000 may include a battery 1005 located at a proximal end of handpiece 1001. Surgical instrument 1000 may be a powered, e.g., motorized tack delivery device. Hand grip 1002 may be slidably mounted onto handpiece 1001.

In use, an operator may move hand grip 1002 proximally or distally along handpiece 1001. Hand grip 1002 allows an operator to have enhanced grip along any desired portion of handpiece 1001. Hand grip 1002 may have a substantially ring shaped profile, but may be modified to any shape or size to suit an operator's preference. Hand grip 1002 may have a friction fit, interference fit, or the like, with handpiece 1001. Hand grip 1002 may be removed from handpiece 1001 and interchanged with another hand grip 1002 (e.g., of different size or shape) to suit an operator's preference.

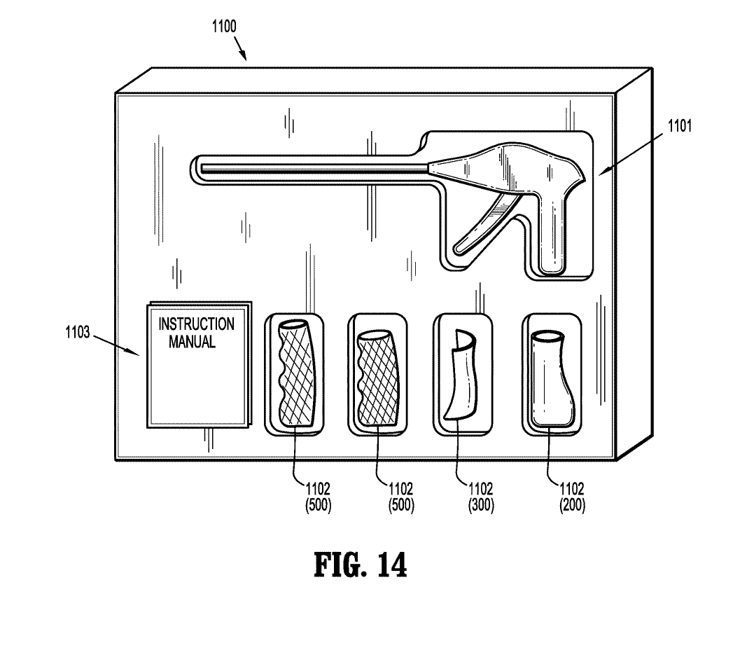

With reference to FIG. 14, a kit is shown and generally designated as 1100. Kit 1100 includes a surgical instrument 1101, at least one hand grip 1102, and an instruction manual 1103 for operating surgical instrument 1101 and using/replacing hand grips 1102. Surgical instrument 1101 is illustrated in FIG. 14. It is contemplated and within the scope of the present disclosure that the surgical instrument of kit 1100 may be any of the aforementioned surgical instruments. Likewise, hand grip 1102 may be any of the aforementioned handgrips, as shown in FIG. 14 (e.g., hand grips 200, 300, and 500 are explicitly shown). Kit 1100 may include one or more surgical instruments 1101 and one or more hand grips 1102 for use during surgical procedures. It should be appreciated that kit 1100 may include other items (not shown) as well.

It should be appreciated that all of the described embodiments may be custom sized, molded and/or fitted for any clinician based on hand size or hand preference. All of the described embodiments may be configured for right hands, left hands, or for both left and right hands. Moreover, all of the described handle grips may be formed from any conformable, flexible, rigid, or semi-rigid material, e.g., rubber, metal, metal alloy, plastic, polymer, or the like. Any of the embodiments described herein may be used separately from and/or in combination with each other, where practical.

Likewise, it should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The embodiments described with reference to the attached drawings are presented only to demonstrate certain examples of the disclosure. Other elements, steps, methods, and techniques that are insubstantially different from those described above and/or in the appended claims are also intended to be within the scope of the disclosure.

What is claimed is:

1. A handheld surgical instrument, comprising:
a housing;
a handle and a trigger mounted on the housing; and
an elongated tubular portion extending distally from the housing;
a hand grip having a top surface, a bottom surface, and at least one opening disposed therebetween;
wherein the handle of the handheld surgical instrument is insertable into the at least one opening of the hand grip for engagement therewith; and
wherein when the hand grip is engaged to the handle of the handheld surgical instrument, the hand grip affects a distance defined between the handle and the trigger.

2. The handheld surgical instrument of claim 1, wherein the hand grip is selectively engageable with the trigger of the handheld surgical instrument.

3. The handheld surgical instrument of claim 2, wherein when the hand grip is engaged with the trigger of the handheld surgical instrument, the hand grip affects the distance defined between the handle and the trigger.

4. The handheld surgical instrument of claim 1, wherein the hand grip includes finger grips for placement of fingers therein.

5. The handheld surgical instrument of claim 1, wherein the hand grip has at least one protuberance or at least one indentation to enhance grip.

6. The handheld surgical instrument of claim 1, wherein the at least one opening is a cavity extending through the top surface of the hand grip to the bottom surface of the hand grip.

7. The handheld surgical instrument of claim 1, wherein the at least one opening is snap fitted with a back portion of the handle.

8. The handheld surgical instrument of claim 1, wherein the at least one opening is a slot extending from the top surface of the hand grip to the bottom surface of the hand grip; and
wherein the slot slidably receives a protrusion disposed along the length of the handle of the handheld instrument.

9. A handheld surgical instrument, comprising:
a housing;
a handle movably mounted on the housing;
an elongated tubular portion extending distally from the housing; and
a trigger movably mounted on the housing.

10. The handheld surgical instrument of claim 9, wherein the handle is pivotably connected to the housing such that the handle can pivot about the housing from a first position to a second position.

11. The handheld surgical instrument of claim 9, wherein the handle includes an inner handle fixedly mounted to the housing;
an outer handle rotatably mounted on the inner handle, the outer handle having a non-uniform shape; and
wherein the outer handle rotates about the inner handle from a first gripping position to a second gripping position.

12. The handheld surgical instrument of claim 9, further comprising:
a hand grip including a hand grip body having a top surface, a bottom surface, and at least one opening disposed therebetween;
wherein the handle of the handheld surgical instrument is insertable into the at least one opening of the hand grip for engagement therewith; and
wherein when the hand grip is engaged to the handle of the handheld surgical instrument, the hand grip affects a distance defined between the handle and a trigger.

13. The surgical instrument of claim 12, wherein the hand grip is selectively engageable with the trigger of the handheld surgical instrument.

14. The surgical instrument of claim 13, wherein when the hand grip is engaged to the trigger of the handheld surgical instrument, the hand grip affects the distance defined between the handle and the trigger.

15. A handheld surgical instrument, comprising:
a handpiece;
a shaft assembly extending distally from the handpiece; and
a hand grip slidably disposed over the handpiece, wherein the hand grip is movable from a proximal end portion of the handpiece to a distal end portion of the handpiece.

16. A handheld surgical instrument kit, comprising:
a handheld surgical instrument, including:
a housing;
a handle and a trigger mounted on the housing; and
an elongated tubular portion extending distally from the housing;
at least two hand grips, each having a top surface, a bottom surface, and at least one opening disposed therebetween, wherein each hand grip is engageable with a handle of the handheld surgical instrument; and
wherein when a selected one of the at least two hand grips is engaged to the handle of the handheld surgical instrument, the selected hand grip affects a distance defined between the handle and the trigger.

17. The kit of claim 16, wherein at least one of the at least two hand grips is engageable with the trigger of the handheld surgical instrument.
